(19) United States
(12) Patent Application Publication
(10) Pub. No.: US 2008/0081912 A1
(43) Pub. Date: Apr. 3, 2008

(54) Near-infrared absorptive image-forming composition, ink and electrophotographic toner using the same, and inkjet-recording method, electrophotographic-recording method and near-infrared-ray-reading method using those

(75) Inventors: Toru Harada, Minami-ashigara-shi (JP); Yoshihiro Jimbo, Minami-ashigara-shi (JP); Yoshimitsu Arai, Ashigarakami-gun (JP)

Correspondence Address: BUCHANAN, INGERSOLL & ROONEY PC, POST OFFICE BOX 1404, ALEXANDRIA, VA, 22313-1404, US

(73) Assignee: FUJIFILM Corporation, Tokyo (JP)

(21) Appl. No.: 11/905,274

(22) Filed: Sep. 28, 2007

(30) Foreign Application Priority Data
Sep. 29, 2006 (JP) 2006-268357
Mar. 15, 2007 (JP) 2007-066974

(51) Int. Cl. C07D 213/02; C07D 209/56

(52) U.S. Cl. 546/276.7; 548/427

(57) ABSTRACT

A near-infrared absorptive image-forming composition, which contains a dye of formula (I):

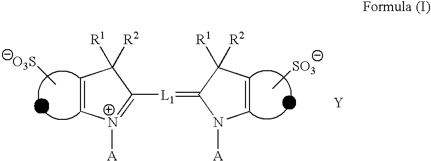

wherein L₁ is a methine chain composed of 7 methine groups, and L₁ may have a substituent but the substituent is not an amino group; R¹, R² and A each independently are an alkyl or cycloalkyl group, which may be substituted; B is a group of atoms necessary for forming an aromatic hydrocarbon ring or aromatic heteroring, and the ring formed with B may be substituted; Y is a cation necessary for keeping the charge balance in the molecule, but Y may not be present when Y is unnecessary for the charge balance; and R¹ and R² may bond to each other, to form a ring; an ink and an electrophotographic toner, each containing the composition; and an inkjet-recording method, an electrophotographic-recording method, and a near-infrared-ray-reading method.

### FIELD OF THE INVENTION

The present invention relates to a near-infrared absorptive image-forming composition, preferably a near-infrared absorptive image-forming composition containing a cyanine dye or its associated substance. The present invention also relates to an ink and electrophotographic toner using the composition; and to an inkjet-recording method, electrophotographic-recording method, and near-infrared-ray-reading method, using the composition, ink or toner.

### BACKGROUND OF THE INVENTION

In general, a code pattern, such as a bar code or an OCR character pattern, which can be read by an optical means, is provided in many cases, as a means for reading out, by an optical means, data described in securities or analogues thereof, such as a stock certificate, a bond, a check, a gift certificate, a public lottery ticket, or a commutation ticket. As code patterns wherein readout by an optical means is used, bar codes are widely used mainly in managing systems of physical distribution, or the like. For example, bar codes are widely used as optical data carriers, such as JAN codes for POS (point-of-sales) systems, delivery vouchers, vouchers or slips for goods or cargos, bar code tags for the delivery of goods. Furthermore, in recent years, two-dimensional codes making it possible to have a larger data capacity and print marks at a higher density have been spreading, examples of such codes including Data Code, Veri Code, Code 1, Maxi Code, and QR Code. Moreover, a method using a dot pattern is also known (see Japanese Patent No. 3706385).

A light from a light source which is utilized to read, by an optical means, out these conventional code patterns is mainly a light emitting diode or a semiconductor laser having an emission wavelength near 650 nm, 800 nm or 950 nm. Such a code pattern can be detected using, for example, a CCD sensor, but the wavelength region of light from the light source is restricted. Thus, currently, code patterns are printed with an ink using carbon black, which has an absorption band in the visible region, or an ink of cyan/green-series having absorption characteristics in the red/infrared wavelength region.

Examples of the method for printing bar codes include a typographic method, an offset printing method, a flexographic method, a gravure printing method, and a silkscreen printing method. These methods are mainly applied to mass printing, so-called source marking. Other examples of the method for printing bar codes include a dot impact method, a thermal transfer method, a direct thermal method, an electrophotographic method, and an inkjet printing method. These methods are mainly applied to one-by-one printing, so-called in-store marking, or to the production of a small lot of data code labels.

In particular, the inkjet recording method has been rapidly spreading, since costs for materials therefor are inexpensive, high-speed recording can be attained, and noise is hardly made in the recording.

The above-mentioned visible data codes give a restriction onto design on printed matters and various other restrictions, such as a reduction in a printing area, to printed matters, and therefore, demand that those restrictions should be removed has been increasing. Furthermore, in order to prevent forgery of securities or analogues thereof, the following attempt has been made: An ink which does not have any absorption band in the visible region is printed to make a data code transparent, thereby making design for printed matters free, overcoming a reduction in the printing area for printing a code pattern, and making it difficult to judge or distinguish the code pattern with the naked eye.

As one of such attempts for making a code pattern transparent (invisible), known is a method of using an ink which mainly absorbs infrared rays outside the visible ray region, thereby to form an infrared absorptive pattern (see JP-A-7-70496 (“JP-A” means unexamined published Japanese patent application) or JP-A-8-143853). The conventional technique of making a code pattern transparent, however, can attain a high infrared absorptivity, but has the following problems: That an infrared ray absorption pattern is not yet made sufficiently invisible, since the pattern has absorptivity also in the visible region; and that when the invisible pattern and a visible image are intermixed, they cannot be sufficiently distinguished from each other, since the infrared ray absorptivity is insufficient. Furthermore, proposed is also the use of a cyanine dye as an improvement in the technique of making a code pattern transparent (see Japanese Patent No. 3114293); however, fastness to light and to humidity and heat are poor. Furthermore, known are a laked cyanine dye in the form of dispersed solid fine-particles (see JP-A-8-333519), a solid fine-particle dispersion of a cyanine compound (see JP-A-8-245902), and a cyanine dye having a pyridyl group (see JP-A-10-231435). However, those dyes and compounds are insufficient in light fastness, humidity and heat resistance, and water resistance.

Further, a toner containing an infrared absorbent is also studied (see JP-A-2006-78888). A toner containing a near-infrared absorbent can be used as an invisible toner, and can be used to form an invisible image for forming, for example, the above-mentioned code pattern. However, the toner disclosed in the above-mentioned publication is insufficient in invisibility and fastness (see JP-A-2006-78888).

### SUMMARY OF THE INVENTION

The present invention resides in a near-infrared absorptive image-forming composition, which comprises a dye represented by the following formula (I):

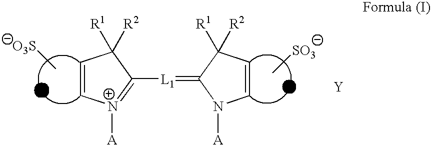

wherein L₁ represents a methine chain composed of 7 methine groups, in which L₁ may have a substituent but the substituent is not an amino group; R¹, R² and A each independently represent an alkyl or cycloalkyl group, which may have a substituent; B represents a group of atoms necessary for forming an aromatic hydrocarbon ring or a group of atoms necessary for forming an aromatic heteroring, and the ring formed by use of B may have a substituent; Y represents a cation necessary for keeping the charge balance in the molecule, in which Y may not be present when Y is unnecessary for the charge balance; and R¹ and R² may bond to each other, to form a ring.

Further, the present invention resides in an ink, which comprises the near-infrared absorptive image-forming composition.

Further, the present invention resides in an inkjet-recording method, which comprises: forming a near-infrared absorptive image, by using the ink.

Further, the present invention resides in an electrophotographic toner, which comprises the near-infrared absorptive image-forming composition.

Further, the present invention resides in an electrophotographic-recording method, which comprises: forming a near-infrared absorptive image, by using the electrophotographic toner.

Further, the present invention resides in a near-infrared-ray-reading method, which comprises: reading a near-infrared absorptive image formed by use of the near-infrared absorptive image-forming composition.

Other and further features and advantages of the invention will appear more fully from the following description.

### DETAILED DESCRIPTION OF THE INVENTION

According to the present invention, there is provided the following means:

### (1) A near-infrared absorptive image-forming composition, comprising a dye represented by formula (I):

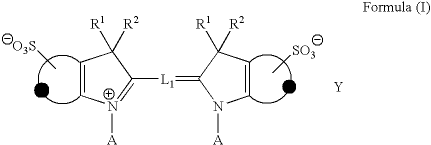

wherein L₁ represents a methine chain composed of 7 methine groups, in which L₁ may have a substituent but the substituent is not an amino group; R¹, R² and A each independently represent an alkyl or cycloalkyl group, which may have a substituent; B represents a group of atoms necessary for forming an aromatic hydrocarbon ring or a group of atoms necessary for forming an aromatic heteroring, and the ring formed by use of B may have a substituent; Y represents a cation necessary for keeping the charge balance in the molecule, in which Y may not be present when Y is unnecessary for the charge balance; and R¹ and R² may bond to each other, to form a ring;

### (2) The near-infrared absorptive image-forming composition according to Item (1), wherein the dye represented by formula (I) is a dye represented by formula (II):

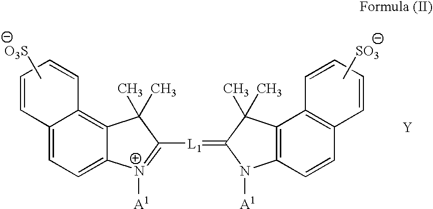

wherein L₁ and Y have the same meanings as in the formula (I); and A¹ represents a sulfoalkyl group;

### (3) The near-infrared absorptive image-forming composition according to Item (1) or (2), wherein the dye represented by formula (I) or (II) forms a substance associated to each other;

### (4) An ink, comprising the near-infrared absorptive image-forming composition according to any one of Items (1) to (3);

(5) An inkjet-recording method, comprising: forming a near-infrared absorptive image, by using the ink according to Item (4);

### (6) An electrophotographic toner, comprising the near-infrared absorptive image-forming composition according to any one of Items (1) to (3);

(7) An electrophotographic-recording method, comprising: forming a near-infrared absorptive image, by using the electrophotographic toner according to Item (6); and
(8) A near-infrared-ray-reading method, comprising: reading a near-infrared absorptive image formed by use of the near-infrared absorptive image-forming composition according to any one of Items (1) to (3).

The present invention is description in detail in the below.

The near-infrared absorptive image-forming composition of the present invention contains the dye represented by formula (I). The composition preferably has an absorption maximum in the wavelength region of 750 to 1,100 nm. The wavelength at which the absorption maximum is shown (the absorption maximum wavelength) may be one which is exhibited when the dye forms a substance associated to each other. Further, with respect to the absorption spectrum of the near-infrared absorptive image-forming composition of the present invention, it is preferred that secondary absorptions are less generated in the visible region (400 to 600 nm); and it is more preferred that there is substantially no absorption in the visible region (herein, “substantially no absorption” means such a degree of absorption that cannot be observed with the naked eye). More specifically, the reflection density (Dv) at an absorption wavelength of 450 nm is preferably ⅕ or less, more preferably 1/7 or less, and particularly preferably 1/10 or less of the reflection density (Dm) at the absorption maximum wavelength.

In order to obtain a preferred absorption waveform, the near-infrared absorptive image-forming composition of the present invention may be made into a composition in which the above-mentioned dye is dissolved in water, a solvent, or the like. To improve light fastness and humidity-and-heat resistance, it is preferred to make the dye into an association state (hereinafter, the dye in this state may be referred to as an “associated dye”); and it is more preferred to use a dye in the state of association containing a J-aggregate.

The associated dye forms a so-called J-band, and exhibits a sharp peak in its absorption spectrum. With respect to association of dyes and J-bands, detailed descriptions can be found, for example, in Photographic Science and Engineering, Vol. 18, pages 323-335 (1974). The absorption maximum of dye in a J-aggregation state shifts to the longer wavelength side (red shift) than the absorption maximum of dye in a solution state. Thus, a judgment whether the dye contained in the near-infrared absorptive image-forming composition is in an aggregation state (association state) or not, can be made by absorption maximum measurement.

In the present invention, the definition of a dye in an association state (i.e., an associated dye) is as follows: That is, the absorption maximum wavelength (λma) of a dye dissolved in water is measured with an absorption wavemeter (trade name: UV-3100Pc) manufactured by Shimadzu Corp.; and the absorption maximum wavelength (λmb) of the dye made into its associated (aggregated) substance (for example, by converting the dye into a lake pigment, as will be explained in the below), is measured with the same wavemeter; and when the λmb is larger by 30 nm or more than the λma, the resultant dye made into the association (aggregate) is called to as a dye in an association state (i.e., an associated dye). When a dye as it is (without receiving any treatment) is not easily dissolved in water, it is allowable to dissolve the dye into water before the dye is laked, followed by measurement of the λma. Further, practically, the absorption maximum wavelength λmb after made into an association (aggregate) is measured, after the dye is used in the form of an ink to be printed out to record an image or character. Further, as a more favorable association state, the associated dye is preferably having the difference between {the absorption maximum wavelength (λmb) of the dye at an association state} and {the absorption maximum wavelength (λma) at the dissolved state of the dye in water}, i.e. (λmb−λma), of 40 nm or more, and more preferably having the difference of 50 nm or more.

The near-infrared absorptive image-forming composition of the present invention contains a dye represented by formula (I), and preferably contains an associated dye thereof:

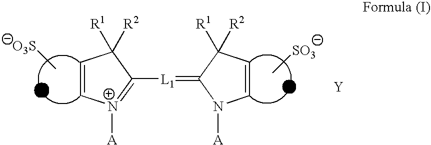

In the formula, L₁ represents a methine chain composed of 7 methine groups, in which L₁ may have a substituent but the substituent is not an amino group. Herein, the “amino group” means to include a primary amino group, a secondary amino group, and a tertiary amino group, each of which includes a cyclic amino group. R¹, R² and A each independently represent an alkyl or cycloalkyl group, which may have a substituent; B represents a group of atoms necessary for forming an aromatic hydrocarbon ring or a group of atoms necessary for forming an aromatic heteroring, and the ring formed by use of B may have a substituent. Y represents a cation necessary for keeping the charge balance in the molecule, in which Y may not be present when Y is unnecessary for the charge balance, and the same is applied to the Y in formula (II). R¹ and R² may bond to each other, to form a ring.

Examples of the aromatic hydrocarbon ring formed by use of the B, include a benzene ring and a naphthalene ring. The aromatic heteroring is preferably a ring wherein at least one ring-constituting atom is a nitrogen atom, an oxygen atom or a sulfur atom, and is more preferably such a ring selected from a 5- to 10-membered ring. The ring may be condensed with another ring (such as an aliphatic ring, an aromatic ring, or a heteroring), or may have a substituent. Examples of the aromatic heteroring include a pyridine ring, a dibenzofuran ring, and a carbazole ring.

Examples of the substituent of the methine chain composed of 7 methine groups represented by L₁, include an alkyl group, a cycloalkyl group, an alkenyl group, a cycloalkenyl group, a halogen atom, an aryl group, a heterocyclic group, OR⁰, or SR⁰, in which R⁰ represents a hydrogen atom, an alkyl group, a cycloalkyl group, an alkenyl group, a cycloalkenyl group, or an aryl group. The substitution position of the substituent is preferably a central position (i.e., a meso-position). Parts of the methine chain may be linked to each other, or substituents on the methine chain may bond to each other, thereby to form a 5- or 6-membered ring, respectively.

In the present invention, the alkyl group as represented by each of R¹, R², A and R⁰ may be linear or branched, and may have a substituent. The cycloalkyl group as represented by each of R¹, R², A and R⁰ may have a substituent. Among those groups, in the present invention, a linear or branched alkyl group is preferred. The number of carbon atoms of the alkyl group is preferably 1 to 20, more preferably 1 to 8, and particularly preferably 1 to 4. The number of carbon atoms of the cycloalkyl group is preferably 3 to 20, more preferably 1 to 8. Examples of the alkyl group and the cycloalkyl group include methyl, ethyl, propyl, isopropyl, butyl, t-butyl, cyclopropyl, cyclohexyl, and 2-ethylhexyl.

Examples of the substituent of the alkyl group and cycloalkyl group include a halogen atom (a fluorine atom, a chlorine atom, a bromine atom, or a iodine atom), an alkyl group, a cycloalkyl group, an alkenyl group, a cycloalkenyl group, an aryl group, a heterocyclic group, —OR¹⁰, —COR¹¹, —COOR¹², —OCOR¹³, —NR¹⁴R¹⁵, —NHCOR¹⁶, —CONR¹⁷R¹⁸, —NHCONR¹⁹R²⁰, —NHCOOR²¹, —SR²², —SO₂R²³, —SO₂OR²⁴, —NHSO₂R²⁵, or —SO₂NR²⁶R²⁷. R¹⁰ to R²⁷ each independently represent a hydrogen atom, an alkyl group, a cycloalkyl group, an alkenyl group, a cycloalkenyl group, an aryl group, or a heterocyclic group. In the case where R¹² of —COOR¹² is a hydrogen atom (i.e., —COOR¹² is a carboxyl group) and in the case where R²⁴ of —SO₂OR²⁴ is a hydrogen atom (i.e., —SO₂OR²⁴ is a sulfo group), the hydrogen atom in each case may be dissociated or the group may be in the state of a salt.

Examples of such a substituted alkyl group include 2-hydroxyethyl, 2-carboxyethyl, 2-methoxyethyl, 2-diethylaminoethyl, 3-sulfopropyl, and 4-sulfobutyl.

In the present invention, the aryl group described as the above-mentioned substituent is preferably a phenyl group or a naphthyl group, each of which may be further substituted with a substituent. Examples of the substituent include the same substituents exemplified as the substituent in the above-mentioned alkyl group, and further include a nitro group and a cyano group. Examples of the substituted aryl group include 4-carboxyphenyl, 4-acetamidophenyl, 3-methanesulfoneamidophenyl, 4-methoxyphenyl, 3-carboxyphenyl, 3,5-dicarboxyphenyl, 4-methanesulfonamidophenyl, and 4-butanesulfonamidophenyl.

In the present invention, the heterocyclic group described as the above-mentioned substituent may have a substituent. It is preferable that the heterocycle of the heterocyclic group is a 5- or 6-membered ring. The heterocycle may have an aliphatic ring or an aromatic ring or another heterocycle condensed thereto. Examples of the heterocycle (including the condensed ring) include a pyridine ring, a pyperidine ring, a furan ring, a furfran ring, a thiophene ring, a pyrrole ring, a quinoline ring, a morpholine ring, an indole ring, an imidazole ring, a pyrazole ring, a carbazole ring, a phenothiazine ring, a phenoxazine ring, an indoline ring, a thiazole ring, a pyrazine ring, a thiadiazine ring, a benzoquinoline ring, and a thiadiazole ring. The substituent on the heterocycle has the same meaning as the substituent of the aryl group described above.

Preferable examples of the cation represented by Y include alkali metal ions (e.g., Li⁺, Na⁺, K⁺), alkali earth metal ions (e.g., Mg²⁺, Ca²⁺, Ba²⁺, Sr²⁺), transition metal ions (e.g., Ag⁺, Fe⁺, Co²⁺, Ni²⁺, Cu²⁺, Zn²⁺), other metal ions (e.g., Al³⁺), ammonium ion, triethyl ammonium ion, tributyl ammonium ion, pyridinium ion, and tetrabutyl ammonium ion; and particularly preferred examples are polyvalent metal ions, such as alkali earth metal ions (e.g., Mg²⁺, Ca²⁺, Ba²⁺, Sr²⁺), transition metal ions (e.g., Ag⁺, Fe⁺, Co²⁺, Ni²⁺, Cu²⁺, Zn²⁺), and other metal ions (e.g., Al³⁺).

When the group represented by R¹ or R² is an alkyl group, the alkyl group is preferably a lower alkyl group having 1 to 3 carbon atoms. It is also preferred that, in that case, R¹ and R² are linked to each other, to form a ring having 5 or 6 carbon atoms.

When the group represented by R¹ or R² is a cycloalkyl group, the cycloalkyl group preferably has 3 to 8 carbon atoms, more preferably 5 to 6 carbon atoms.

A is preferably an alkyl group having a sulfo group (e.g., sulfoethyl, sulfopropyl, sulfobutyl).

The dye represented by formula (I) is preferably a dye represented by formula (II), and is more preferably an associated dye thereof:

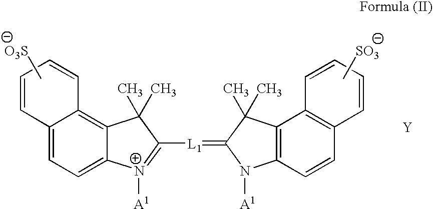

In the formula, L₁ and Y have the same meanings as in the formula (I); A¹ represents a sulfoalkyl group (the sulfoalkyl group is an alkyl group having a sulfo group, and preferred examples thereof include sulfoethyl, sulfopropyl and sulfobutyl). Y in formula (II) is in particular preferably Mg²⁺, Ca²⁺ or Zn²⁺.

Specific examples of the dye represented by formula (I) (or (II)) according to the present invention are shown below, but the present invention is not meant to be limited thereto. Herein, ‘Me’ represents a methyl group, ‘Ph’ represents a phenyl group.

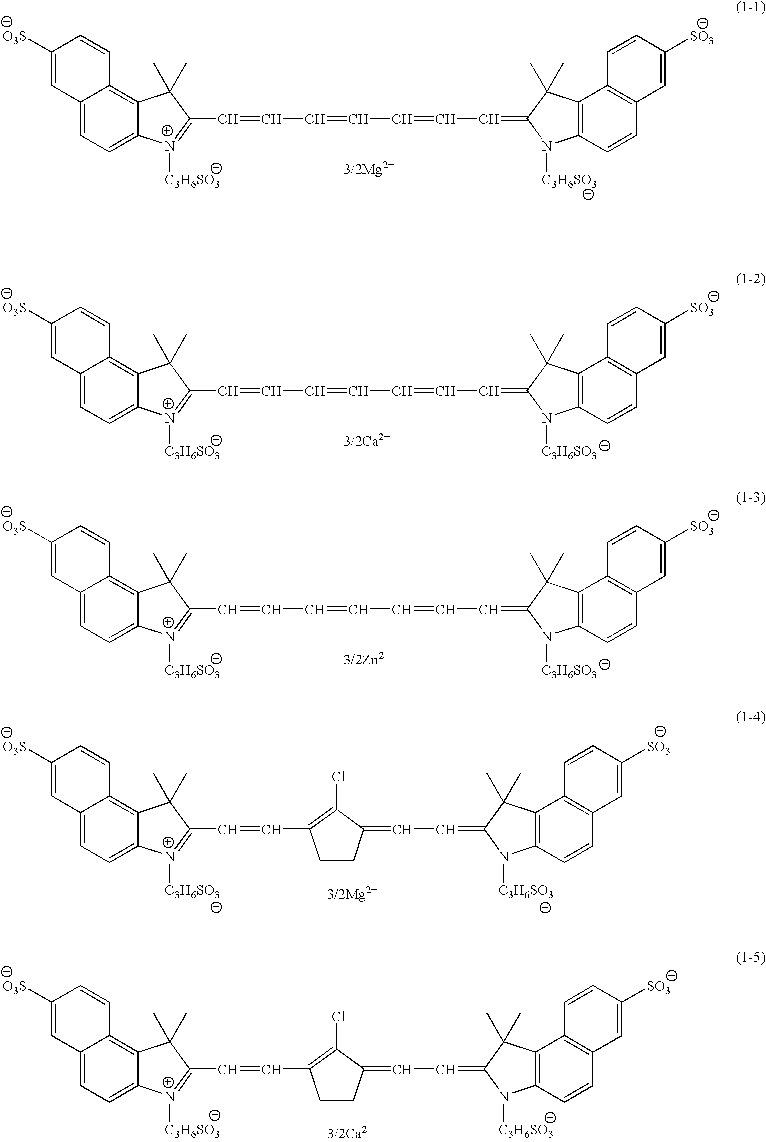

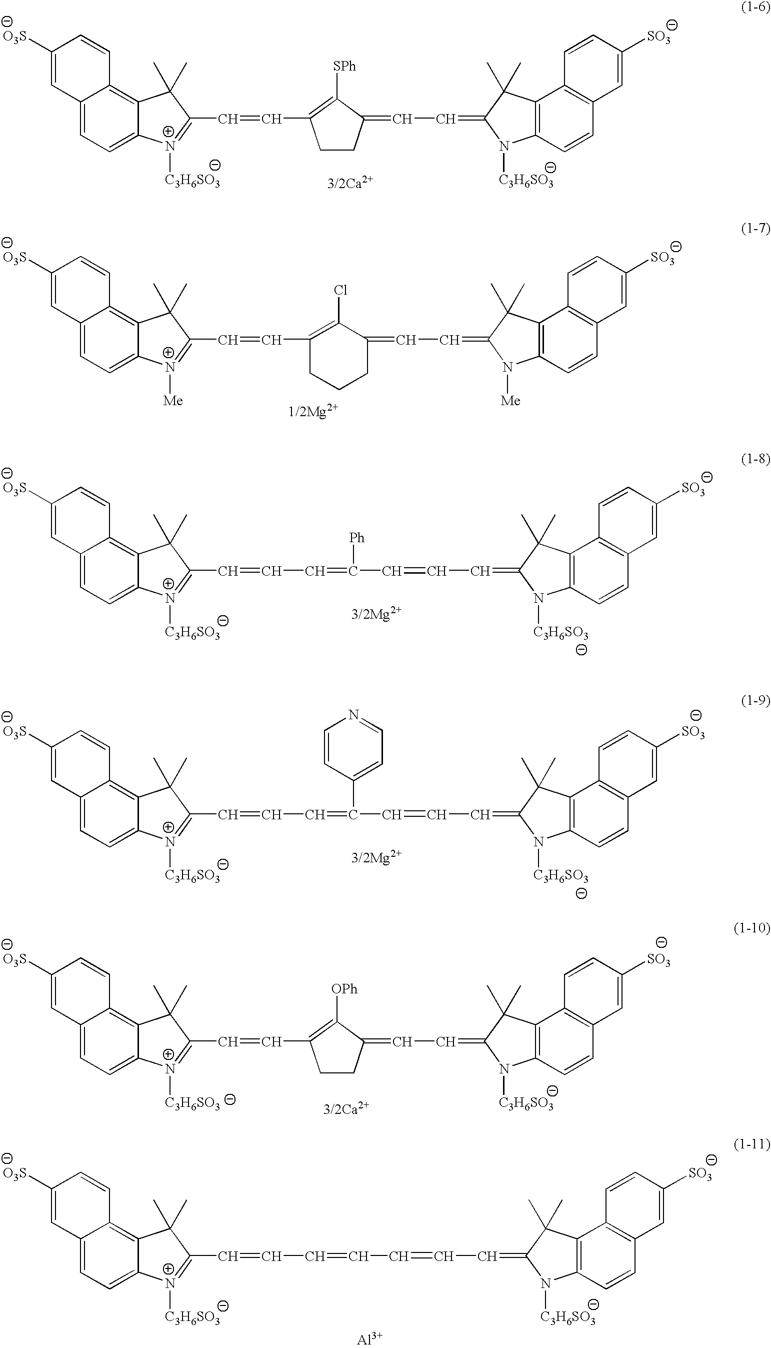

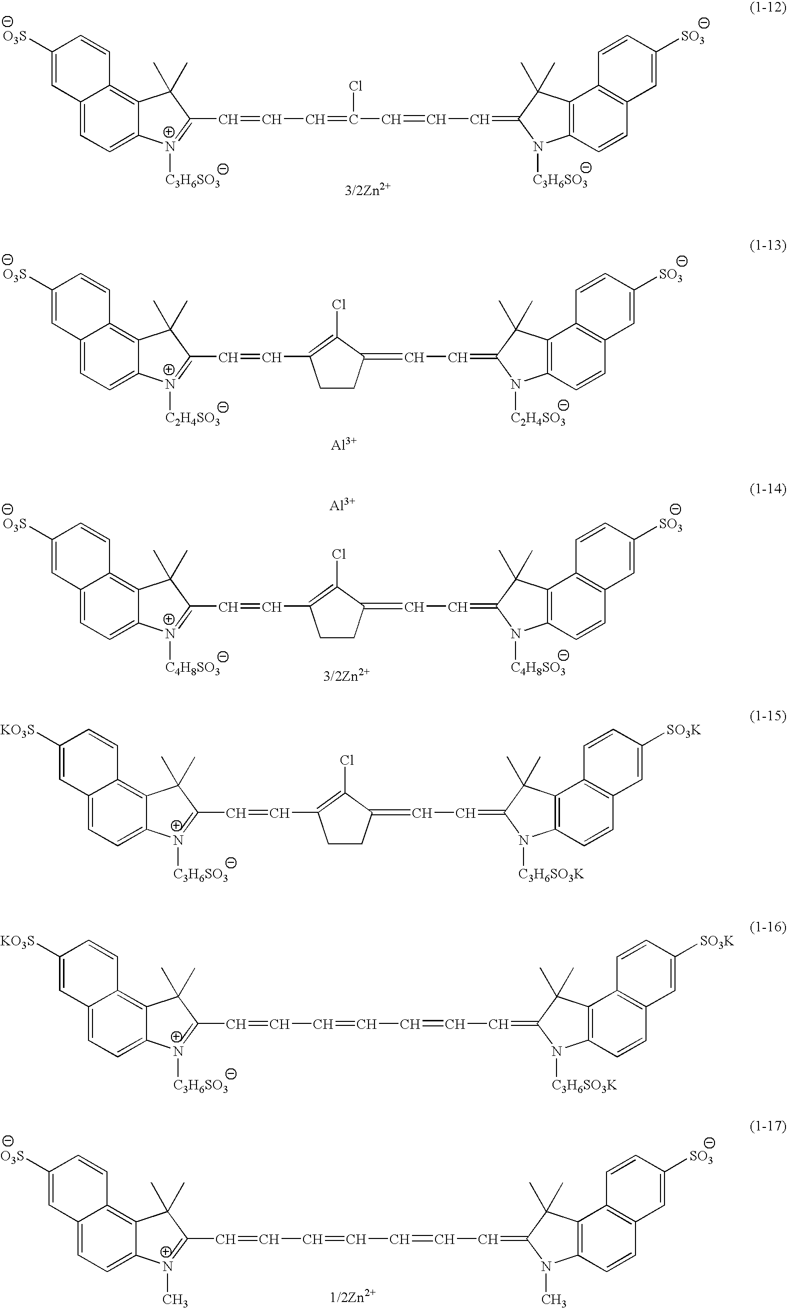

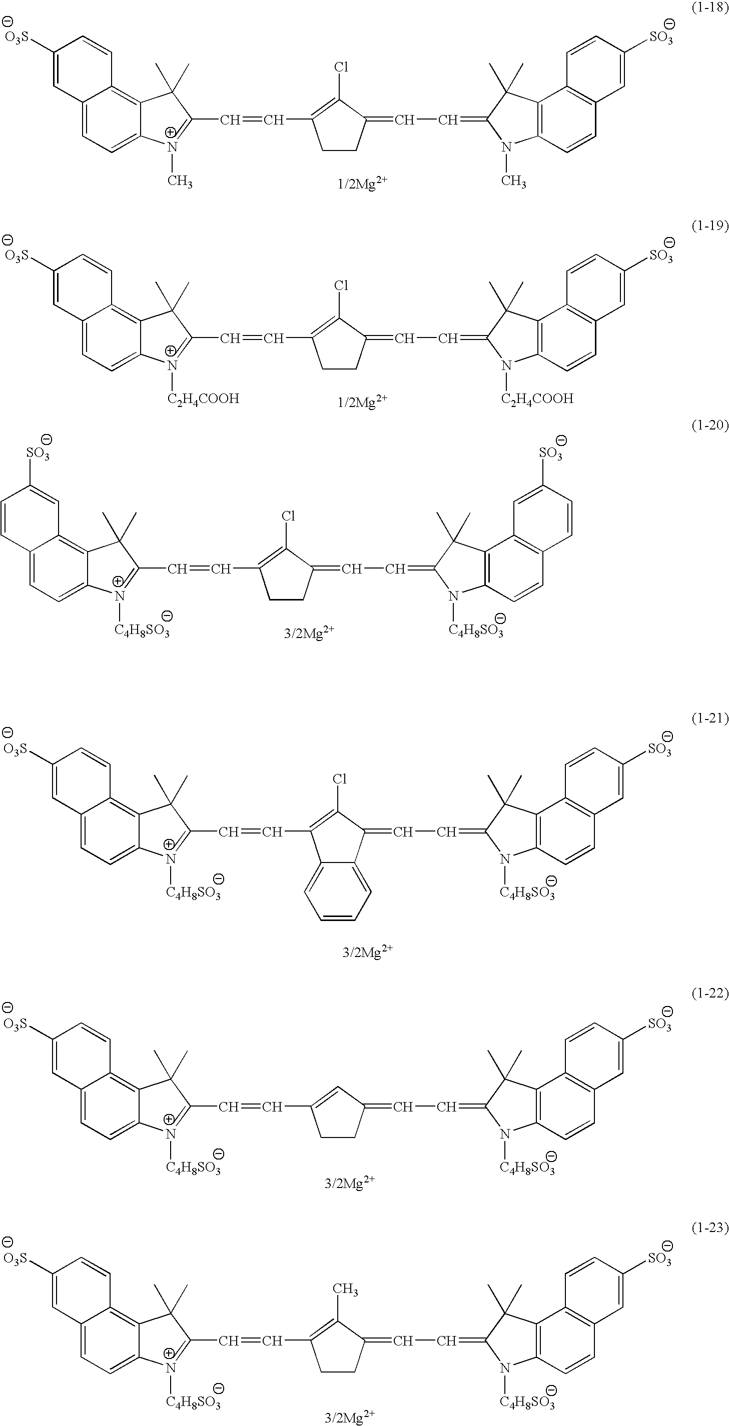

The dyes represented by formula (I) or (II) can be synthesized, by referring, for example, to the references by F. M. Harmer, “Heterocyclic Compounds Cyanine Dyes and Related Compounds”, John Wiley & Sons, New York, London (1964); by D. M. Sturmer, “Heterocyclic Compounds—Special Topics in Heterocyclic Chemistry”, Chapter 18, Section 14, pp. 482-515, John Wiley & Sons, New York, London (1977); “Rodd's Chemistry of Carbon Compounds”, 2nd Ed., Vol. IV, Part B, Chapter 15, pp. 369-422, Elsevier Science Publishing Company Inc., New York (1977); JP-A-6-313939, and JP-A-5-88293.

As described above, in the near-infrared absorptive image-forming composition of the present invention, the dye represented by formula (I) (herein, the “dye represented by formula (I)” will also include the meaning of a dye represented by formula (II) as a preferred dye thereof, unless otherwise specified) is preferably made into its associated dye. The associated dye can be formed, by dissolving the dye represented by formula (I) into water, and adding gelatin or a salt (such as, barium chloride or calcium chloride) to the solution, to cause association of the dye in the water. Preferably, the dye is converted to a lake pigment, thereby making the dye into an associated dye in which the associated substances are dispersed as solid fine-particles. The conversion to the lake pigment may also be attained, by adding an aqueous solution of a metal salt (such as, magnesium chloride, zinc chloride, or barium chloride) to an aqueous solution of the dye represented by formula (I). Conversely, to the above-mentioned aqueous solution of a metal salt may be added an aqueous solution of the dye represented by formula (I). Herein, embodiments for deriving a lake pigment have been described. The dye represented by formula (I) may be a dye converted to a lake pigment, or a dye before (or not) converted to a lake pigment. Out of the two, the former dye converted to a lake pigment is preferred.

The amount to be added of the compound represented by formula (I) in the near-infrared absorptive image-forming composition of the present invention can be appropriately adjusted, and the amount is preferably 0.1 to 30% by mass, more preferably 0.5 to 10% by mass in the near-infrared absorptive image-forming composition.

The solid fine-particle dispersion of the lake pigment (or lake pigment derivative) of the dye represented by formula (I) is minutely described in, for example, “Ganryou Bunsan Gijyutsu—Hyoumenshori to Bunsanzai no Tsukaikata oyobi Bunsanseihyouka—(Technology of Pigment Dispersion—Surface Treatment and Way of Using Dispersant and Evaluation of Dispersion Properties—)” published by Kabushiki-kaisha Gijutsu Jouhoukyoukai; “Ganryou no Jiten (Encyclopedia of Pigment)” published by Kabushiki-kaisha Asakura Shoten; and “Saisin [Ganryou Bunsan] Jitsumu Nouhau-Jireisyu (The Newest [Pigment Dispersion] Practical Know-how and Case Examples” published by Kabushiki-kaisha Gijutsu Jouhoukyoukai. In order to obtain the dispersion of solid fine-particles, a usual dispersion machine can be used. Examples of the dispersion machine include a ball mill, a vibration ball mill, a planetary ball mill, a sand mill, a colloid mill, a jet mill, and a roller mill. JP-A-52-92716 and WO88/074794 disclose such dispersion machines. It is preferable to employ a medium dispersion machine of upright or lateral type.

The dispersion process may be carried out in the presence of an appropriate medium (e.g., water, an alcohol, cyclohexanone, 2-methoxy-1-methylethyl acetate). It is preferable that a surfactant for dispersion is used together. As the surfactant for dispersion, an anionic surfactant (as disclosed in JP-A-52-92716 and WO88/074794) is preferably used. An anionic polymer, a nonionic surfactant or a cationic surfactant may optionally be used.

If necessary, powder of the dye represented by formula (I) may be obtained, by dissolving the dye into an appropriate solvent, and adding a poor solvent to the solution, to make the dye into fine particles. The aforementioned surfactant for dispersion may be used in this case, as well. Alternatively, fine-particles of the dye can be deposited, by dissolving the dye in a solvent by adjusting the pH value of the solution, and then changing the pH. It is preferred that the fine particles also constitute the above-mentioned associated dye.

In the case where the associated dye is in the form of fine particles (or microcrystals), the average particle diameter is preferably 1,000 μm or less, more preferably 0.001 μm to 100 μm, particularly preferably 0.005 μm to 50 μm.

When the near-infrared absorptive image-forming composition of the present invention is prepared, an ordinary surfactant or dispersing agent for pigment can be added to the composition, in order to improve the dispersibility of a resultant lake pigment (or a thus-derived lake pigment) of the dye represented by formula (I). Such a dispersant to be used can be selected from a wide variety of compounds, and examples thereof include phthalocyanine derivatives (e.g., EFKA-745 (trade name), a commercial product manufactured by EFKA); organosiloxane polymers (e.g., KP341 (trade name), produced by Shin-etsu Chemical Industry Co., Ltd.); (meth)acrylic (co)polymers (e.g., Polyflow No. 75, No. 90 and No. 95 (trade names), produced by Kyoei-sha Yushi Kagaku Kogyo); cationic surfactants (e.g., W001 (trade name), produced by Yusho); nonionic surfactants, such as polyoxyethylene lauryl ether, polyoxyethylene stearyl ether, polyoxyethylene oleyl ether, polyoxyethylene octyl phenyl ether, polyoxyethylene nonyl phenyl ether, polyethylene glycol dilaurate, polyethylene glycol distearate, and sorbitan fatty acid esters; anionic surfactants, such as W004, W005 and W 017 (trade names) (produced by Yusho); polymeric dispersants, such as EFKA-46, EFKA-47, EFKA-47EA, EFKA POLYMER 100, EFKA POLYMER 400, EFKA POLYMER 401, and EFKA POLYMER 450 (trade names, produced by Morishita Industries Co., Ltd.), and Disperse Aid 6, Disperse Aid 8, Disperse Aid 15, and Disperse Aid 9100 (trade names, produced by San-Nopco); various kinds of Solsperse dispersants, such as Solsperse 3000, 5000, 9000, 12000, 13240, 13940, 17000, 24000, 26000, and 28000 (trade names, produced by Zeneka); ADEKA PLURONIC L31, F38, L42, L44, L61, L64, F68, L72, P95, F77, P84, F87, P94, L101, P103, F108, L121, P-123 (trade names, product by Asahi Denka Co., Ltd.), and Isonet S-20 (trade name, produced by Sanyo Chemical Industries Co., Ltd.).

Any one of the dispersants may be properly selected for use, and examples thereof include cationic surfactants, fluorine-containing surfactants, polymer dispersants, and the like.

In addition, the graft copolymers, as described in JP-A-10-254133, containing, as the main chain unit thereof, a monomer moiety having a particular acid amido group or a monomer moiety having a quaternary ammonium salt group, have excellent function in finely dispersing the pigment, and may be used as the dispersant. By using the graft copolymer above, it is possible to disperse the pigment finely while the consumption of energy and period of time is reduced, as well as to prevent aggregation and sedimentation of the dispersed pigment with the lapse of time, and to keep the dispersion stable for an extended period of time.

These dispersants may be used singly or as a mixture of two or more thereof. The amount of dispersant to be added in the composition of the present invention, is preferably about 1 to 150 parts by mass, per 100 parts by mass of the lake pigment (or lake pigment derivative) of the compound represented by formula (I).

The ink of the present invention comprises the above-mentioned near-infrared absorptive image-forming composition, and contains at least one dye represented by formula (I). It is preferred to make the ink of the present invention into an inkjet recording ink or printing ink to which a medium is incorporated.

The ink of the present invention can be prepared, by dissolving and/or dispersing the dye represented by formula (I) in a lipophilic medium or an aqueous medium. Preferably, an aqueous medium is used. The ink of the present invention, however, means to include an ink not containing any medium. As required, another additive(s) may be incorporated. Examples of such an additive include a drying inhibitor (a wetting agent), a fading-preventing agent, an emulsion stabilizer, a penetration accelerator, an ultraviolet ray absorber, a preservative, a mildew-proofing agent, a pH-adjusting agent, a surface-tension-adjusting agent, an antifoaming agent, a viscosity-adjusting agent, a dispersing agent, a dispersion stabilizer, a rust-proofing agent, a chelating agent, and the like. In the case of water-soluble ink, these various additives are directly added to the ink liquid. In the case that an oil-soluble dye is used in a dispersion form, the additives are generally added to a dye dispersion after its preparation but they may be added to an oily phase or an aqueous phase at the time of preparation.

The drying inhibitor is preferably used for the purpose of preventing clogging from being occurred at an ink injection port of a nozzle to be used in the inkjet recording system, in which the clogging may be caused due to drying of the inkjet ink.

The drying inhibitor is preferably a water soluble organic solvent having a vapor pressure lower than water. Specific examples thereof include polyhydric alcohols represented by ethylene glycol, propylene glycol, diethylene glycol, polyethylene glycol, thiodiglycol, dithiodiglycol, 2-methyl-1,3-propanediol, 1,2,6-hexanetriol, acetylene glycol derivatives, glycerin, trimethylol propane, and the like; lower alkyl ethers of polyhydric alcohols, such as ethylene glycol monomethyl (or monoethyl)ether, diethylene glycol monomethyl (or monoethyl)ether, triethylene glycol monoethyl (or monobutyl)ether, or the like; heterocyclic compounds such as 2-pyrrolidone, N-methyl-2-pyrrolidone, 1,3-dimethyl-2-imidazolidinone, N-ethylmorpholine, and the like; sulfur-containing compounds, such as sulfolane, dimethylsulfoxide, 3-sulfolene, and the like; polyfunctional compounds, such as diacetone alcohol, diethanolamine, and the like; and urea derivatives. Of these, polyhydric alcohols, such as glycerin, diethylene glycol, and the like are more preferable. The drying inhibitor may be used singly or two or more of them may be used in combination. The drying inhibitor is preferably contained in the ink in an amount of 10 to 50% by mass.

The penetration accelerator is preferably used for the purpose of better penetration of the inkjet ink into paper. As the penetration accelerator, alcohols, such as ethanol, isopropanol, butanol, di(tri)ethylene glycol monobutyl ether, 1,2-hexanediol, and the like; sodium laurylsulfate, sodium oleate, nonionic surfactants, and the like can be used. When the penetration accelerator is contained in the ink in an amount of 5 to 30% by mass, sufficient effects are usually exhibited, and it is preferable to use it within a range of addition amount where bleeding of the printed characters or print-through do not occur.

The ultraviolet ray absorber is used for the purpose of enhancing the storability of the resultant image. As the ultraviolet ray absorber, use may be made, for example, of the benzotriazole-based compounds described in JP-A-58-185677, JP-A-61-190537, JP-A-2-782, JP-A-5-197075, JP-A-9-34057, and the like; the benzophenone-based compounds described in JP-A-46-2784, JP-A-5-194483, U.S. Pat. No. 3,214,463, and the like; the cinnamic acid-based compounds described in JP-B-48-30492 (“JP-B” means examined Japanese patent publication), JP-B-56-21141, JP-A-10-88106, and the like; the triazine-based compounds described in JP-A-4-298503, JP-A-8-53427, JP-A-8-239368, JP-A-10-182621, JP-T-8-501291 (“JP-T” means published searched patent publication), and the like; compounds described in Research Disclosure No. 24239; and compounds that absorb ultraviolet ray and emit fluorescent light, so-called fluorescent brightening agents, represented by stilbene-based compounds and benzooxazole-based compounds.

The fading-preventing agent is used for the purpose of enhancing the storability of the resultant image. As the fading-preventing agent, various organic or metal complex-based fading-preventing agents can be used. Examples of the organic fading-preventing agents include hydroquinones, alkoxyphenols, dialkoxyphenols, phenols, anilines, amines, indanes, chromans, alkoxyanilines, heterocyclic compounds, and the like. Examples of metal complex fading-preventing agents include nickel complexes, zinc complexes, and the like. More concretely, use may be made of compounds described in the patents cited in Research Disclosure No. 17643 Chapter VII, items I to J, ibid., No. 15162, ibid., No. 18716, page 650, left column, ibid., No. 36544, page 527, ibid., No. 307105, page 872, ibid., No. 15162; compounds falling within formulas of representative compounds and compound examples described in JP-A-62-215272, pages 127 to 137.

Examples of the mildew-proofing agent include sodium dehydroacetate, sodium benzoate, sodium pyridinethion-1-oxide, ethyl p-hydroxybenzoate, 1,2-benzisothiazolin-3-on, and salts thereof. It is preferable to use the mildew-proofing agent in the ink in an amount of 0.02 to 1.00% by mass.

As the pH-adjusting agent, the above neutralizing agents (organic bases and inorganic alkalis) can be used. For the purpose of enhancing the storage stability of the inkjet ink, the pH-adjusting agent is added such that the inkjet ink preferably has a pH of 6 to 10, and more preferably a pH of 7 to 10.

Examples of the surface-tension-adjusting agent include nonionic, cationic, or anionic surfactants. The surface tension of the inkjet ink of the present invention is preferably from 20 to 60 mN/m, and more preferably from 25 to 45 mN/m. The viscosity of the inkjet ink of the present invention is preferably set to 30 mPa-s or less, more preferably 20 mPa-s or less. Examples of the preferable surfactant include anionic surfactants, such as fatty acid salts, alkylsulfate esters/salts, alkylbenzenesulfonic acid salts, alkylnaphthalenesulfonic acid salts, dialkylsulfosuccinic acid salts, alkylphosphate esters/salts, naphthalenesulfonic acid formalin condensates, polyoxyethylene alkylsulfate esters/salts, and the like; and nonionic surfactants, such as polyoxyethylene alkyl ethers, polyoxyethylene alkyl allyl ethers, polyoxyethylene fatty acid esters, sorbitan fatty acid esters, polyoxyethylene sorbitan fatty acid esters, polyoxyethylene alkylamines, glycerin fatty acid esters, oxyethylene/oxypropylene block copolymers, and the like. Also, SURFYNOLS (trade name, manufactured by Air Products & Chemicals), which is an acetylene-type polyoxyethyleneoxide surfactant, can be preferably used. Moreover, an amine oxide-type amphoteric surfactant, such as N,N-dimethyl-N-alkylamine oxide, and the like, is preferable. Furthermore, surfactants described in JP-A-59-157636, pp. (37)-(38), and Research Disclosure No. 308119 (1989) can also be used.

As the antifoaming agent, fluorine-containing compounds, silicone type compounds, and chelating agents represented by EDTA can be used, if necessary.

As described above, the ink of the present invention preferably contains an aqueous medium. As the aqueous medium, a mixture containing water as a main component and a water-miscible organic solvent as an optional component may be used. Examples of the water-miscible organic solvent include alcohols (e.g., methanol, ethanol, propanol, iso-propanol, butanol, isobutanol, sec-butanol, t-butanol, pentanol, hexanol, cyclohexanol, and benzylalcohol), polyhydric alcohols (e.g., ethylene glycol, diethylene glycol, triethylene glycol, polyethylene glycol, propylene glycol, dipropylene glycol, polypropylene glycol, butylene glycol, hexanediol, pentanediol, glycerin, hexanetriol, and thiodiglycol), glycol derivatives (e.g., ethylene glycol monomethyl ether, ethylene glycol monoethyl ether, ethylene glycol monobutyl ether, diethylene glycol monomethyl ether, diethylene glycol monobutyl ether, propylene glycol monomethyl ether, propylene glycol monobutyl ether, dipropylene glycol monomethyl ether, triethylene glycol monomethyl ether, ethylene glycol diacetate, ethylene glycol monomethyl ether acetate, triethylene glycol monomethyl ether, triethylene glycol monoethyl ether, and ethylene glycol monophenyl ether), amines (e.g., ethanolamine, diethanolamine, triethanolamine, N-methyldiethanolamine, N-ethyldiethanolamine, morpholine, N-ethylmorpholine, ethylenediamine, diethylenetriamine, triethylenetetramine, polyethyleneimine, and tetramethylpropylenediamine), and other polar solvents (e.g., formamide, N,N-dimethylformamide, N,N-dimethylacetamide, dimethyl sulfoxide, sulfolane, 2-pyrrolidone, N-methyl-2-pyrrolidone, N-vinyl-2-pyrrolidone, 2-oxazolidone, 1,3-dimethyl-2-imidazolidinone, acetonitrile, and acetone). These water-miscible organic solvents may be used as a mixture of two or more.

The details of methods of preparing the inkjet-recording ink are described in JP-A-5-148436, JP-A-5-295312, JP-A-7-97541, JP-A-7-82515, JP-A-7-118584, JP-A-11-286637, and the publications of Japanese Patent Applications No. 2000-87539, No. 2000-80259, No. 2000-78491 and No. 2000-203857. These methods can also be utilized in the preparation of the ink of the present invention.

As the ink of the present invention, it is preferable that the compound represented by formula (I) is contained in an amount of 0.2 to 10 mass %, more preferably 0.5 to 9 mass %. The ink of the present invention may contain another dye(s), together with the compound represented by formula (I). In the case where two or more kinds of dyes are used in combination, it is preferable that the total content of the above dye and said another dye(s) falls within the aforementioned range.

Furthermore, in the case where a single-color image is to be formed or even in the case where a full-color image is to be formed, the near-infrared absorptive image-forming composition of the present invention may contain a visible absorptive dye or pigment, which is used in the usual inkjet-recording ink. Alternatively, the ink composed of the near-infrared absorptive image-forming composition of the present invention may be used together with any of those inks for inkjet recording. For the formation of a full-color image, a magenta-tone ink, a cyan-tone ink, and a yellow-tone ink can be used. In addition, for adjustment of color tone, a black-tone ink may also be used.

In accordance with the inkjet-recording method of the present invention, the ink is energized, to form an image on an image-receiving material (e.g., ordinary papers, resin-coated papers, films, electrophotographic papers, clothes, glasses, metals, ceramics, inkjet papers, as described, for example, in JP-A-8-169172, JP-A-8-27693, JP-A-2-276670, JP-A-7-276789, JP-A-9-323475, JP-A-62-238783, JP-A-10-153989, JP-A-10-217473, JP-A-10-235995, JP-A-10-337947, JP-A-10-217597, and JP-A-10-337947).

In forming an image, a latex polymer compound may be used in combination for the purpose of giving glossiness or water resistance or improving the weather resistance. The timing of imparting the polymer latex to the image-receiving material may be before or after imparting the coloring agent or simultaneously with it. Accordingly, the site to which the latex polymer compound is added may be in the image-receiving paper or ink, or a liquid material composed of the latex polymer compound singly may be prepared and used. More specifically, the methods described in JP-A-2002-166638, JP-A-2002-121440, JP-A-2002-154201, JP-A-2002-144696, JP-A-2002-80759, JP-A-2002-187342 and JP-A-2002-172774 can be preferably used.

The recording system for the inkjet-recording method of the present invention is not particularly limited. For example, the inkjet-recording method of the present invention may be used in any of a charge-controlling system of jetting out (ejecting) ink through electrostatic attractive force; a drop-on-demand system (pressure pulse system) of using the oscillation pressure of a piezoelectric device; an acoustic inkjet system of converting an electric signal into an acoustic beam, applying it to ink, and jetting out the ink under radiation pressure; or a thermal inkjet (bubble jet) system of heating ink to form bubbles and utilizing the resulting pressure. The inkjet recording system includes a system of jetting a large number of small-volume drops of ink of low concentration so-called photoink, a system of using multiple inks of substantially the same color which, however, differ in concentration to improve the image quality, and a system of using colorless transparent ink. Recording papers and recording films for use in carrying out inkjet printing are described in JP-A-2003-277662, and the like.

The electrophotographic toner of the present invention comprises the above-mentioned near-infrared absorptive image-forming composition, and preferably at least contains a binder resin and the dye represented by formula (I). It is preferred to use the toner as a toner for optical fixation or an invisible toner.

Herein, the “invisible toner” means a toner for use in decoding an image by use of invisible ray such as infrared ray. When the invisible toner is fixed as a toner image onto a sheet of paper or the like, the toner image may or may not be perceived with the naked eye. Thus, the invisible toner means to include any toner that can form an image which can be read out through invisible light. In other words, the invisible toner means a toner for forming an invisible image, for example, an infrared absorptive pattern, such as a bar code. Even if a coloring agent is added to the toner, the resultant toner can be even called an invisible toner when the added amount of the coloring agent is, for example, 1 mass % or less, which is clearly at such a level that the presence of the coloring agent cannot be perceived. The invisible toner includes one that is to be optically fixed.

The electrophotographic toner of the present invention may contain an ordinary binder resin. The binder resin is preferably composed mainly of a polyester or polyolefin. The following may be used singly or in combination thereof: a copolymer of styrene and acrylic acid or methacrylic acid, polyvinyl chloride, a phenolic resin, an acrylic resin, a methacrylic resin, polyvinyl acetate, a silicone resin, a polyester resin, polyurethane, a polyamide resin, a furan resin, an epoxy resin, a xylene resin, polyvinyl butyral, a terpene resin, a cumarone indene resin, a petroleum resin, a polyether polyol resin, and the like. From the viewpoint of durability, translucency and the like, it is preferred to use a polyester-based resin or a norbornene polyolefin resin. The glass transition point (Tg) of the binder resin used in the toner is preferably within the range of from 50 to 70° C.

If necessary, a charge controlling agent or a wax may be added to the electrophotographic toner of the present invention. Examples of the charge controlling agent include calixarene, nigrosine dyes, quaternary ammonium salts, amino group-containing polymers, metal-containing azo dyes, complex compounds of salicylic acid, phenol compounds, azo chromium compounds, and azo zinc compounds. Alternatively, the toner may include a magnetic material, such as iron powder, magnetite, and ferrite, and the resultant toner can be a magnetic toner.

The wax that can be contained in the electrophotographic toner of the present invention is most preferably an ester wax, polyethylene, polypropylene, or a ethylene/propylene copolymer, but may be another wax. Examples of the aforementioned another wax include polyglycerin wax, microcrystalline wax, paraffin wax, carnauba wax, sazole wax, montanic acid ester wax, deoxidized (or deacidified) carnauba wax; unsaturated fatty acids, e.g. brassidic acid, eleostearic acid, parinaric acid, palmitic acid, stearic acid, and montanic acid; saturated alcohols, e.g. stearyl alcohol, aralkyl alcohol, behenyl alcohol, carnaubyl alcohol, ceryl alcohol, melissyl alcohol, and long-chain-alkyl alcohols whose alkyl moiety is longer than that of the above-mentioned alcohols; polyhydric alcohols, e.g. sorbitol; fatty acid amides, e.g. linoleic acid amide, oleic acid amide, and lauric acid amide; saturated fatty acid bisamides, e.g. methylenebisstearic acid amide, ethylenebiscapric acid amide, ethylenebislauric acid amide, and hexamethylenebisstearic acid amide; unsaturated fatty acid amides, e.g. ethylenebisoleic acid amide, hexamethylenebisoleic acid amide, N,N′-dioleyladipic acid amide, and N,N′-dioleylsebacic acid amide; aromatic bisamides, e.g. m-xylenebisstearic acid amide, and N,N′-distearylisophthalic acid amide; metal salts of fatty acids (generally called as metallic soap), e.g. calcium stearate, calcium laurate, zinc stearate, and magnesium stearate; waxes obtained by graft-copolymerizing an aliphatic hydrocarbon wax with a vinyl-based monomer, e.g. styrene or acrylic acid; partially-esterified compounds of a polyhydric alcohol and a fatty acid, e.g. behenic acid monoglyceride; and methyl ester compounds containing a hydroxyl group obtained by, for example, hydrogenating a vegetable fat or oil.

The electrophotographic toner of the present invention can be produced, by using, for example, a kneading-pulverization method or a wet granulation method, which is a usually utilized method. Examples of the wet granulation method that can be used include a suspension polymerization method, an emulsion polymerization method, an emulsion polymerization aggregation method, a soap-free emulsion polymerization method, a non-aqueous dispersion polymerization method, an in-situ polymerization method, an interfacial polymerization method, and an emulsion dispersion granulation method.

When the electrophotographic toner of the present invention is produced by the kneading pulverization method, the target toner can be obtained by: thoroughly mixing a binder resin, the dye represented by formula (I), a wax, a charge controlling agent, and another additive(s) if any, in a mixer, such as a HENSCHEL mixer or a ball mill; melt-kneading the resultant mixture with a heat kneader, such as a heating roll, a kneader, or an extruder, to disperse or dissolve the aforementioned agents in the resins which have been compatibilized or dissolved each other; cooling down the resultant, to solidify the same; and pulverizing and classifying the resultant particles, thereby to give the toner. The dye represented by formula (I) may be added before or after the melt-kneading. To improve the dispersibility of the dye represented by formula (I), a master batch treatment may be conducted.

In the electrophotographic toner of the present invention, the content of the dye represented by formula (I) is not particularly limited, and is preferably from 0.1 to 30% by mass, more preferably from 0.5 to 10% by mass.

The electrophotographic toner of the present invention preferably has a volume average particle diameter (D50v) in the range of 3 to 10 μm, and more preferably in the range of 4 to 8 μm. The toner preferably has a ratio (D50v/D50p) of the volume average particle diameter (D50v) to the number average particle diameter (D50p) in the range of 1.0 to 1.25. Thus, by using such a toner having a small particle diameter and a narrow diameter distribution, it is possible to suppress unevenness in chargeability of the toner, to form an image with a reduced level of fogging, and to improve fixability of the toner. Further, the aforementioned toner can improve reproducibility of fine lines and reproducibility of dots with respect to the resulting image formed with the toner.

The electrophotographic-recording method of the present invention can be conducted in accordance with an ordinary embodiment in a copying machine, a printer, a printing machine, or the like. For example, an image can be formed as follows. First, a positive or negative uniform electrostatic charge is given onto the surface of a photoconductive insulator on a photosensitive drum. After this electrifying step, for example, a laser beam is radiated onto the resultant photoconductive insulator surface, to erase the electrostatic charge on the insulator surface partially, thereby forming an electrostatic latent image corresponding to image data. Next, for example, fine powder of a developing agent called a toner (or an electrostatic image developing toner) is caused to adhere onto the latent image region, in where the electrostatic charge remains, on the photoconductive insulator surface, thereby making the latent image visible to convert into a toner image. In order to make the thus-obtained toner image into an image on a printed matter, the toner image is generally transferred electrostatically onto a recording medium, such as recording paper, and then the toner image is fixed onto the recording medium.

The method for fixing the toner image after the transferring is, for example, a method of melting the toner in a pressing manner, a heating manner, or a manner using pressing and heating together, and then solidifying/fixing the toner image; or a method of radiating optical energy onto the toner to melt the toner, and then solidifying/fixing the toner image.

According to the present invention, there can be provided a near-infrared absorptive image-forming composition excellent in spectral characteristics, and an ink and an electrophotographic toner using the composition. Further, according to the present invention, there can also be provided a near-infrared absorptive dye-containing image-forming composition, which is excellent in invisibility, light fastness, humidity-and-heat resistance, and water resistance; an ink and an electrophotographic toner using the composition; and an inkjet-recording method, an electrophotographic-recording method and a near-infrared-ray-reading method, using the ink, the toner, or the composition.

The near-infrared absorptive image-forming composition of the present invention has substantially no absorptivity in the visible region. An image formed by use of the ink or electrophotographic toner using the composition is excellent in light fastness, humidity-and-heat resistance, and water resistance. Further, the near-infrared image-forming method, the inkjet-recording method, and the electrophotographic-recording method of the present invention make it possible to form a favorable invisible image from the above-mentioned excellent near-infrared absorptive image-forming composition, or the ink or the electrophotographic toner using the composition, thereby realizing a highly reliable readout of a near-infrared image.

The present invention will be described in more detail based on the following examples, but the invention is not intended to be limited thereto.

### EXAMPLES

### Example 1

### Comparative Example 1

### Preparation of Ink 1

The following components were made into a dispersion over 3 hours, by using an Eiger Motor Mill (trade name, manufactured by Eiger Japan K.K.), thereby to yield a composition sample (Sample No. 1) according to the present invention containing the above-shown Exemplified dye (1-4).

| | |
|---|---|
| DEMOL SNB (trade name, manufactured by KAO Corporation) | 0.5 g |
| Exemplified dye (1-4), as shown above | 5.0 g |
| H₂O | 50.0 g |

The following components were mixed with each other, followed by stirring at 35° C. for 1 hour. Then, the resultant mixture was filtrated through a micro-filter of average pore diameter 5 μm under reduced pressure, to yield an ink sample (Ink 1) of the present invention. As the remnant, to the following components was added extra pure water (specific resistance of 18 M·Ω or more), to adjust the total amount to 100 parts by mass.

| | |
|---|---|
| Composition Sample 1, as mentioned in the above | 40.3 parts by mass |
| Glycerin | 10 parts by mass |
| Triethylene glycol | 10 parts by mass |
| Triethylene glycol monobutyl ether | 10 parts by mass |
| Triethanolamine | 0.2 parts by mass |
| OLFINE E1010 (trade name)*¹ | 1 parts by mass |

*¹Acetylene glycol-based surfactant, manufactured by Nissin Chemical Industry Co., Ltd.

### Formation of Printed Matters 1a and 1b

The Ink 1 was filled into an ink cartridge for a black ink for an inkjet printer (trade name: PM-A700, manufactured by SEIKO EPSON Corporation), to print a solid image onto a photo glossy paper (PM photographic paper <gloss> (trade name: KA420PSK, EPSON), manufactured by SEIKO EPSON Corporation) under the condition that the set-up color was fixed to “black”, to thereby give a Printed matter 1a.

Separately, the Ink 1 was filled into an ink cartridge for a black ink for an inkjet printer (trade name: PM-A700, manufactured by SEIKO EPSON Corporation), to print bar-code-form microlines of line width 200 μm and line interval of 2 mm onto a photo glossy paper (PM photographic paper <gloss> (trade name: KA420PSK, EPSON), manufactured by SEIKO EPSON Corporation) on the basis of “highly minute color digital standard image data (ISO/JIS-SCID)” under the condition that the set-up color was fixed to “black”, to thereby give a Printed matter 1b.

### Preparation of Inks 2 to 5, and Formation of Printed Matters Using the Inks

Inks 2 to 5 each were prepared in the same manner as the Ink 1, except that one of the exemplified dyes as shown in Table 1 was used. Further, Printed matters 2a to 5a and 2b to 5b were formed in the same manner as the Printed matters 1a and 1b, respectively, except that the Ink 1 was changed to any of the Inks 2 to 5, respectively.

### Formation of Printed Matters Using Ink C1 for Comparison

Printed matters C1a and C1b were formed in the same manner as the Printed matters 1a and 1b, respectively, except that a genuine black ink (Ink C1) for an inkjet printer (trade name: PM-A700, manufactured by SEIKO EPSON Corporation) was used instead of the Ink 1.

### Formation of Printed Matters Using Ink C2 for Comparison

Printed matters C2a and C2b were formed in the same manner as the Printed matters 1a and 1b, respectively, except that an inkjet printer (trade name: PX-G930, manufactured by SEIKO EPSON Corporation) and a genuine ink (Ink C2) for the printer were used, under the conditions that the set-up color was fixed to black and a gloss optimizer was set into an OFF-mode.

With respect to color density in the visible region, using a reflection densitometer (trade name: X-Rite 310 TR, manufactured by Gretag Macbeth), measurement was made of the density (Dm) in the maximum reflection density region in the visible region of each of the Printed matters 1a, 2a, 3a, 4a, 5a, C1a, and C2a, in each of which the solid image was formed. The results are shown in Table 1. As is apparent from the results, it can be understood that the printed matters formed by use of the ink of the present invention have a quite low reflection density in the visible region and thus a highly invisible image can be obtained according to the present invention.

Separately, using a spectrophotometer (trade name: UV-3100Pc, manufactured by Shimadzu Corp.), measurement was made of the absorption maximum wavelength (λma) of each of the exemplified dyes dissolved in water (in which, with respect to the absorption of the dye in water, 1 mg of the dye was dissolved into 100 mL of water before the dye was laked, and then the absorption maximum was measured). Apart from this measurement, using the same device, measurement was made of the absorption maximum wavelength (λmb) of each of the Printed matters 1a to 5a. As is apparent from Table 1, with respect to each of the exemplified dyes, the absorption maximum wavelength thereof was observed at a site of sufficiently longer side in the printed matter than in the solution, and thus a favorable associated dye was formed.

With respect to readout (readability) of data in the region outside of the visible region, it was tested and evaluated whether or not the data in each of the Printed matters 1b, 2b, 3b, 4b, 5b, C1b, and C2b was able to be read out, by use of: a bar code reader (trade name: THLS-6000 & TBR-6000, manufactured by Tohken Co., Ltd.), in which a laser source of emission wavelength 780 nm was used as a light source; a light source of an infrared emitting diode (trade name: GL480, manufactured by SHARP Corporation, emission wavelength peak of 950 nm); and a light receiving section of a photodiode (trade name: PD413PI, manufactured by SHARP Corporation, emission wavelength peak of 960 nm).

The test of readout of the data was repeatedly carried out 10 times for each of the printed matters.

With respect to the 10 readout operations, the results were evaluated in the following manners: the case where the data were correctly read out every time is designated to as “∘”, the case where the data were unable to be read out one or two times is designated to as “Δ”, and the case where the data were unable to be read out three or more times is designated to as “x”. The results are shown in Table 1.

TABLE 1

| | | Printed | | | Printed | | |
|---|---|---|---|---|---|---|---|
| Ink | Dye | matter | Dm | λmb | matter | Readability | λma |
| 1 | 1-4 | 1a | 0.29 | 940 nm | 1b | ∘ | 840 nm |
| 2 | 1-1 | 2a | 0.20 | 920 nm | 2b | ∘ | 780 nm |
| 3 | 1-6 | 3a | 0.25 | 910 nm | 3b | ∘ | 850 nm |
| 4 | 1-7 | 4a | 0.20 | 930 nm | 4b | ∘ | 813 nm |
| 5 | 1-9 | 5a | 0.26 | 920 nm | 5b | ∘ | 800 nm |
| C1 | | C1a | 2.19 | | C1b | x | |
| C2 | | C2a | 2.18 | | C2b | ∘ | |

As is apparent from the results in Table 1, it can be understood that, in the case of using the near-infrared absorptive image-forming composition of the present invention, a favorable bar code can be printed, which is quite high in invisibility, which causes no affection on a visible image, and which realizes a quite highly reliable readout.

### Example 2

### Comparative Example 2

### Preparation of Ink 11

The following components were made into a dispersion over 3 hours, by using an Eiger Motor Mill, thereby to yield a composition sample (Sample No. 11) according to the present invention.

| | |
|---|---|
| DEMOL SNB (trade name, manufactured by KAO Corporation) | 0.5 g |
| Exemplified dye (1-1), as shown above | 5.0 g |
| H₂O | 50.0 g |

Further, the following components were added to the Composition Sample 11, and to the resultant mixture was added deionized water to make the total volume to 1 L. The resultant solution was then stirred for 1 hour while heated at 30 to 40° C. Then, the solution was adjusted to pH 9 with a 10-mol/L aqueous KOH solution, followed by filtration with a micro-filter of average pore diameter 5 μm under reduced pressure, to give an Ink 11.

| | | |
|---|---|---|
| Diethylene glycol | 20 | g |
| Glycerin | 120 | g |
| Diethylene glycol monobutyl ether | 230 | g |
| 2-Pyrrolidone | 80 | g |
| Triethanolamine | 17.9 | g |
| Benzotriazole | 0.06 | g |
| SURFYNOL TG (trade name)*² | 8.5 | g |
| PROXEL XL2 (trade name)*³ | 1.8 | g |

*²Surfactant, manufactured by Air Products
*³Preservative, manufactured by Zeneca K.K.

### Preparation of Inks 12 and 13

Ink samples 12 to 13 each were prepared in the same manner as the Ink sample 11, except that the dye (1-1) was changed to one as shown in Table 2.

Ink samples C11 to C13 each were prepared in the same manner as the Ink sample 11, except that the dye (1-1) was changed to one as shown in Table 2. The dyes (a), (b) and (c) for comparison are those described in Japanese Patent No. 3114293, JP-A-8-333519, and JP-A-10-231435, respectively. When the dye (1-1) was changed to any of the dyes for comparison, the mol number of the dye to be added was made equal to each other in the ink solution.

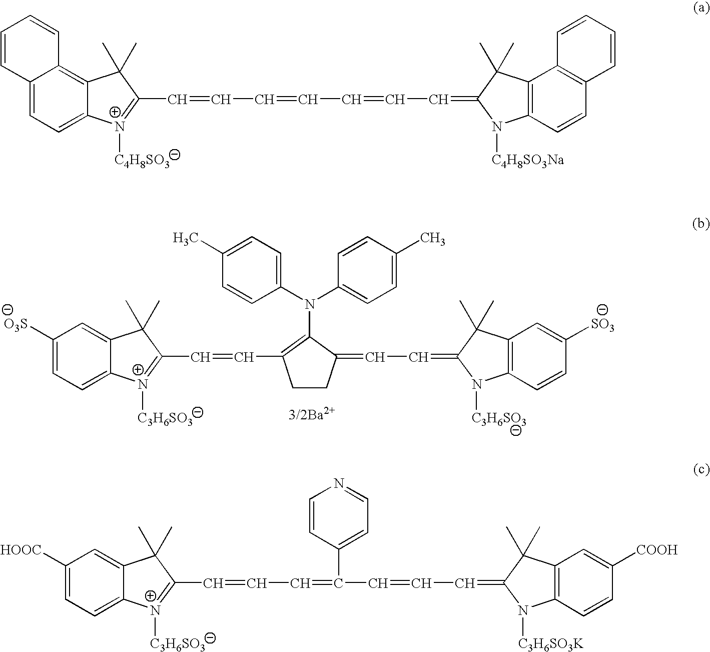

### Image Recording and Evaluations

With respect to the Ink samples 11, 12, 13, C11, C12, and C13, the following tests and evaluations were made. The results are shown in Table 2.

In Table 2, the evaluations of “Invisibility”, “Light fastness”, “Humidity-and-heat resistance”, and “Water resistance” were made, after an image was recorded onto a photo glossy paper (PM photographic paper <gloss> (trade name: KA420PSK, EPSON), manufactured by SEIKO EPSON Corporation), with any of the ink samples for inkjet ink, with an inkjet printer (trade name: PM-700C, manufactured by SEIKO EPSON Corporation).

### Invisibility

With respect to invisibility, the case where the value (Dv/Dm) obtained by dividing the reflection density (Dv) at 450 nm by the reflection density (Dm) at the absorption maximum wavelength was less than 1/10 is designated to as “A”; the case where the value (Dv/Dm) was 1/10 or more and less than ⅕ is designated to as “B”; and the case where the value (Dv/Dm) was ⅕ or more is designated to as “C”. In this way, the invisibility was evaluated into one of the three ranks. Further, Table 2 also shows the value of the absorption maximum wavelength λmb obtained by using the above-mentioned spectrophotometer (trade name: UV-3100Pc) to measure the reflection spectrum of each of the PM photographic papers after the image was recorded.

### Light Fastness

Using a weather meter (trade name: ATLAS C. 165, manufactured by Atlas Electric Device), radiation of xenon light (85,000lx) was given to each of the photo glossy papers on which the image was formed, for 3 day; and then, using the above-mentioned spectrophotometer (trade name: UV-3100Pc), measurement was made of the image density before the radiation of the xenon light and the image density after the radiation, at each of three points on the paper. An evaluation was made on the basis of: (The dye remaining ratio)=(the density after the radiation)/(the density before the radiation). At the three points, the reflection densities before the radiation were 1, 1.5, and 2.0, respectively.

The case where the dye remaining ratio was 70% or more at each of the three points of the three densities before the radiation, is designated to as “A”; the case where the ratio was less than 70% at one or two out of the three points, is designated to as “B”; and the case where the ratio was less than 70% at all of the three points, is designated to as “C”. In this way, the light fastness was evaluated into one of the three ranks.

### Humidity-and-Heat Resistance

Each of the photo glossy papers on which the image was formed, was allowed to stand still, for 7 days, in a box where the temperature and the humidity were set to 60° C. and 90%, respectively. After the standing, the image density was measured with the spectrophotometer (trade name: UV-3100 Pc), at each of three points on the paper. An evaluation was made on the basis of: (The dye remaining ratio)=(the density after the radiation)/(the density before the radiation). At the three points, the reflection densities before the radiation were 1, 1.5, and 2.0, respectively.

The case where the dye remaining ratio was 70% or more at each of the three points of the three densities before the radiation, is designated to as “A”; the case where the ratio was less than 70% at one or two out of the three points, is designated to as “B”; and the case where the ratio was less than 70% at all of the three points, is designated to as “C”. In this way, the humidity-and-heat resistance was evaluated into one of the three ranks.

### Water Resistance

Each of the photo glossy papers on which the image was formed, was dried at room temperature for 1 hour, and then immersed into deionized water for 10 seconds. The paper was then naturally dried at room temperature. After the immersion, the image density was measured with the spectrophotometer (trade name: UV-3100 Pc). An evaluation was made on the basis of: (The dye remaining ratio)=(the density after the immersion)/(the density before the immersion). The reflection density measured before the immersion at that point on the paper, was 1.0.

The case where the dye remaining ratio was 70% or more, is designated to as “A”; the case where the ratio was 40% or more and less than 70%, is designated to as “B”; and the case where the ratio was less than 40%, is designated to as “C”. In this way, the water resistance was evaluated into one of the three ranks.

### Readability

The readability (readout of data) of each of the samples was measured and evaluated in the same manner as in Example 1 and Comparative example 1.

TABLE 2

| | | | | | Humidity- | | |
|---|---|---|---|---|---|---|---|
| | | | | Light | and-heat | Water | |
| Ink sample | Dye | Invisibility | λmb | fastness | resistance | resistance | Readability |
| 11 | 1-1 | A | 920 nm | A | A | A | ∘ |
| 12 | 1-4 | A | 940 nm | A | A | A | ∘ |
| 13 | 1-7 | A | 930 nm | A | A | A | ∘ |
| C11 | a | C | 830 nm | C | B | B | ∘ |
| C12 | b | B | 920 nm | C | B | A | Δ |
| C13 | c | B | 920 nm | B | B | B | ∘ |

As is apparent from Table 2, each image recorded by use of the ink of the present invention is excellent in invisibility, and is also excellent in all of light fastness, humidity-and-heat resistance, and water resistance. Further, the results according to the present invention show that the readability of the bar code is also favorable.

Separately, the readability of each of the printed matters after the light fastness test was conducted, was also tested in the same manner as above. As a result, the printed matters formed by use of the Inks 11 to 13 according to the present invention were able to be favorably read out, while the printed matters formed by use of the Inks C11 to C13 for comparison were unable to be read out.

### Example 3

### Comparative Example 3

### Preparation of Toner 101

A mixture of 0.5 g of the DEMOL SNB (trade name: manufactured by KAO Corporation), 5.0 g of the Exemplified dye (1-1), and 50.0 g of ion-exchange water was made into a dispersion over 3 hours, by using an Eiger Motor Mill, and then, from the resultant dispersion, the solid content was collected by filtration, followed by drying, thereby to yield a powdery composition (Powdery composition No. 101) according to the present invention, which contained the dye (1-1).

Further, 37.5 g of styrene, 8.7 g of butyl acrylate, 1.1 g of acrylic acid, 0.5 g of dodecane thiol, 0.2 g of 11,10-diacryloyloxydodecane, and 1.5 g of the Powdery composition 101 were mixed, followed by making the resultant mixture into a dispersion by using the Eiger Motor Mill over 3 hours. Forty grams of the thus-obtained liquid dispersion was slowly added to an aqueous solution in which 0.4 g of the DEMOL SNB (trade name: manufactured by KAO Corporation) was dissolved in 55 g of ion-exchange water, to cause dispersing and emulsifying in a flask. While the resultant dispersed and emulsified product was slowly stirred for 10 minutes to mix the components therein, 5 g of ion-exchange water in which 0.6 g of ammonium persulfate was dissolved was poured into the flask. Then, the inside of the flask was purged with nitrogen, followed by heating till the internal temperature would be 70° C. under stirring. While this state was kept as it was for 5 hours, the emulsion polymerization was continued, to yield an anionic-resin fine-particle liquid dispersion. The resultant liquid dispersion was cooled, and then filtrated, and the resultant solid was washed with ion-exchanged water. The thus-obtained solid was re-dispersed into ion-exchange water, and the dispersion was filtrated. The resultant solid was dried under reduced pressure, and pulverized, to thereby yield a toner sample (Sample No. 101).

### Preparation of Toners 102 to 106

Toner samples 102 to 106 each were prepared in the same manner as the Toner sample 101, except that the dye was changed to one as shown in Table 3.

Using any of the thus-obtained toner samples and a piece of plain paper as a recording medium, a printed matter (specifically, the image of the similar bar code, as the Printed matter 1b in Example 1) was formed, by means of an image-forming device capable of attaining thermal fixation. The image-forming device to be used was one (trade name: Docucentre 402 FS, manufactured by Fuji Xerox Co., Ltd.) equipped with a heat roller as a thermally fixing unit. The invisibility, the light fastness, and the humidity-and-heat resistance were tested and evaluated in the same manner as in Example 2. The evaluation of the amount of toner adhered was made within the range of 0.7±0.05 mg/cm². The results are shown in Table 3.

TABLE 3

| | | | Light | Humidity-and- |
|---|---|---|---|---|
| Sample | Dye | Invisibility | fastness | heat resistance |
| 101 | 1-1 | A | A | A |
| 102 | 1-4 | A | A | A |
| 103 | 1-7 | A | A | A |
| 104 | a | C | C | B |
| 105 | b | B | C | B |
| 106 | c | B | B | B |

As is apparent from Table 3, the toner of the present invention is excellent in all of invisibility, light fastness, and humidity-and-heat resistance of the resultant image recorded. Separately, it was also found out that the readability of a bar code was favorable, in the recording with the toner of the present invention, by use of: a bar code reader (trade name: THLS-6000 & TBR-6000, manufactured by Tohken Co., Ltd.), in which a laser source of emission wavelength 780 nm was used as a light source; a light source of an infrared emitting diode (trade name: GL480, manufactured by SHARP Corporation, emission wavelength peak of 950 nm); and a light receiving section of a photodiode (trade name: PD413PI, manufactured by SHARP Corporation, emission wavelength peak of 960 nm).

Having described our invention as related to the present embodiments, it is our intention that the invention not be limited by any of the details of the description, unless otherwise specified, but rather be construed broadly within its spirit and scope as set out in the accompanying claims.

This non-provisional application claims priority under 35 U.S.C. § 119 (a) on Patent Application No. 2006-268357 filed in Japan on Sep. 29, 2006, and Patent Application No. 2007-066974 filed in Japan on Mar. 15, 2007, each of which is entirely herein incorporated by reference.

What we claim is:

1. A near-infrared absorptive image-forming composition, comprising a dye represented by formula (I): 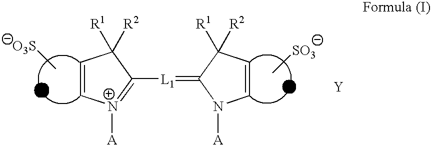
wherein L₁ represents a methine chain composed of 7 methine groups, in which L₁ may have a substituent but the substituent is not an amino group; R¹, R² and A each independently represent an alkyl or cycloalkyl group, which may have a substituent; B represents a group of atoms necessary for forming an aromatic hydrocarbon ring or a group of atoms necessary for forming an aromatic heteroring, and the ring formed by use of B may have a substituent; Y represents a cation necessary for keeping the charge balance in the molecule, in which Y may not be present when Y is unnecessary for the charge balance; and R¹ and R² may bond to each other, to form a ring.

2. The near-infrared absorptive image-forming composition according to claim 1, wherein the dye represented by formula (I) forms a substance associated to each other.

3. The near-infrared absorptive image-forming composition according to claim 1, wherein the dye represented by formula (I) is a dye represented by formula (II): 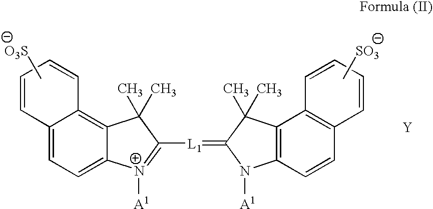
wherein L₁ and Y have the same meanings as in the formula (I); and A¹ represents a sulfoalkyl group.

4. The near-infrared absorptive image-forming composition according to claim 3, wherein the dye represented by formula (II) forms a substance associated to each other.

5. An ink, comprising the near-infrared absorptive image-forming composition according to claim 1.

6. The ink according to claim 5, wherein the dye represented by formula (I) forms a substance associated to each other.

7. The ink according to claim 5, wherein the dye represented by formula (I) is a dye represented by formula (II): 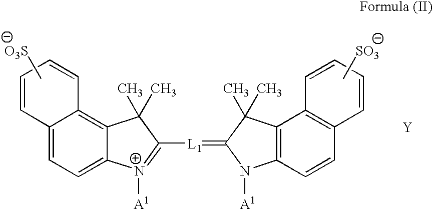
wherein L₁ and Y have the same meanings as in the formula (I); and A¹ represents a sulfoalkyl group.

8. The ink according to claim 7, wherein the dye represented by formula (II) forms a substance associated to each other.

9. An inkjet-recording method, comprising: forming a near-infrared absorptive image, by using the ink according to claim 5.

10. The inkjet-recording method according to claim 9, wherein the dye represented by formula (I) forms a substance associated to each other.

11. The inkjet-recording method according to claim 9, wherein the dye represented by formula (I) is a dye represented by formula (II): 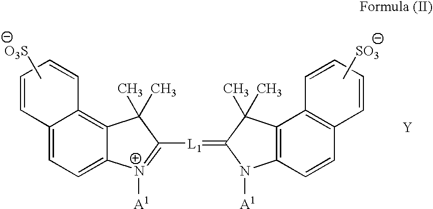
wherein L₁ and Y have the same meanings as in the formula (I); and A¹ represents a sulfoalkyl group.

12. The inkjet-recording method according to claim 11, wherein the dye represented by formula (II) forms a substance associated to each other.

13. An electrophotographic toner, comprising the near-infrared absorptive image-forming composition according to claim 1.

14. The electrophotographic toner according to claim 13, wherein the dye represented by formula (I) forms a substance associated to each other.

15. The electrophotographic toner according to claim 13, wherein the dye represented by formula (I) is a dye represented by formula (II): 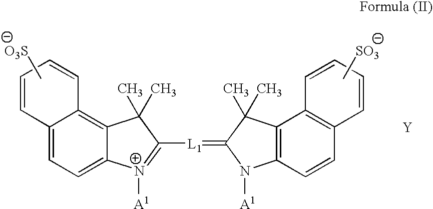
wherein L₁ and Y have the same meanings as in the formula (I); and A¹ represents a sulfoalkyl group.

16. The electrophotographic toner according to claim 15, wherein the dye represented by formula (II) forms a substance associated to each other.

17. An electrophotographic-recording method, comprising: forming a near-infrared absorptive image, by using the electrophotographic toner according to claim 13.

18. The electrophotographic-recording method according to claim 17, wherein the dye represented by formula (I) forms a substance associated to each other.

19. The electrophotographic-recording method according to claim 17, wherein the dye represented by formula (I) is a dye represented by formula (II): 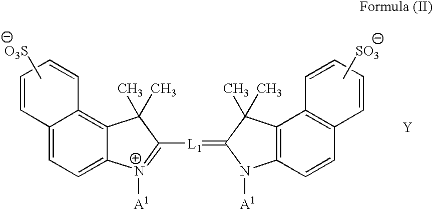
wherein L₁ and Y have the same meanings as in the formula (I); and A^{f} represents a sulfoalkyl group.

20. The electrophotographic-recording method according to claim 19, wherein the dye represented by formula (II) forms a substance associated to each other.

21. A near-infrared-ray-reading method, comprising: reading a near-infrared absorptive image formed by use of the near-infrared absorptive image-forming composition according to claim 1.

22. The near-infrared-ray-reading method according to claim 21, wherein the dye represented by formula (I) forms a substance associated to each other.

23. The near-infrared-ray-reading method according to claim 21, wherein the dye represented by formula (I) is a dye represented by formula (II): 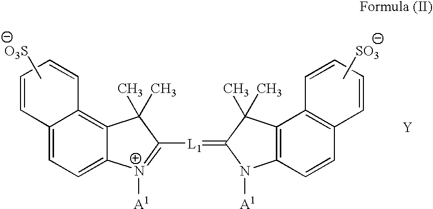
wherein L₁ and Y have the same meanings as in the formula (I); and A¹ represents a sulfoalkyl group.

24. The near-infrared-ray-reading method according to claim 23, wherein the dye represented by formula (II) forms a substance associated to each other.
